(19) United States
(12) Patent Application Publication
(10) Pub. No.: US 2026/0061162 A1
(43) Pub. Date: Mar. 5, 2026

(54) AGENT DELIVERY CATHETER

(71) Applicant: Precision NeuroMed Inc., Woodside, CA (US)

(72) Inventor: Sandeep KUNWAR, Woodside, CA (US)

(21) Appl. No.: 19/314,447

(22) Filed: Aug. 29, 2025

Related U.S. Application Data
(60) Provisional application No. 63/689,601, filed on Aug. 30, 2024.

(51) Int. Cl. A61M 25/00

(52) U.S. Cl. CPC A61M 25/003; A61M 25/0032; A61M 25/0045; A61M 25/0052; A61M 2025/0001; A61M 2025/0031; A61M 2025/0037; A61M 2025/0042; A61M 2025/0063; A61M 2205/0238; A61M 2205/3327; A61M 2205/3331; A61M 2205/3368; A61M 2205/3375; A61M 2210/0693

(57) ABSTRACT

A medical catheter includes a multi-lumen tube comprising a proximal end, a distal end, a first lumen, and a second lumen, the first lumen being sealed at the distal end. A retractable stylet of the catheter is carried in the first lumen of the multi-lumen tube. The stylet is adapted to stiffen the multi-lumen tube during a placement process and is movable relative to the multi-lumen tube. The first lumen being sealed at the distal end prevents the retractable stylet from protruding from the distal end of the multi-lumen tube. A capillary tube of the catheter is associated with the second lumen of the multi-lumen tube. The capillary tube has a distal tip for releasing an agent at the distal tip to a biological tissue. The distal tip of the capillary tube is an exposed portion of the capillary tube that protrudes beyond the distal end of the multi-lumen tube.

### CROSS-REFERENCE TO RELATED APPLICATION

This application claims the benefit of U.S. Provisional Application Ser. No. 63/689,601, filed Aug. 30, 2024, the entire content of which is incorporated by reference herein.

### TECHNICAL FIELD

Embodiments of the invention generally relate to convection-enhanced delivery (CED) therapy, and more particularly to a catheter which is used within the framework of CED therapy.

### BACKGROUND

Convection-enhanced delivery (CED) is a promising technique for delivering therapeutic agents directly to brain tissue. It bypasses the blood-brain barrier, a delicate defense system that restricts the passage of large molecules from the bloodstream into the brain. CED works by infusing drugs through a catheter placed within the brain parenchyma. A pressure gradient is then established, driving the therapeutic agent through the interstitial fluid, bathing a larger volume of tissue compared to traditional diffusion-based methods, with significantly higher concentration of drug.

Despite its advantages, CED faces challenges. Current single-port catheters can limit the distribution of the therapeutic agent, potentially leaving areas of the target tissue untreated. Additionally, achieving a uniform distribution throughout larger tumors or complex anatomical regions can be difficult. Furthermore, air bubbles within the catheter lumen can be a significant concern. Air bubbles can disrupt the flow of the therapeutic agent, leading to uneven distribution and potentially compromising treatment efficacy.

Another major limitation of conventional CED catheters is reflux. Reflux occurs when the infused therapeutic agent flows out of the catheter tip and backs up along the external surface of the catheter, hindering its penetration into the target brain tissue. Reflux can be caused by several factors, including catheter design. Simple, single-port designs may not effectively prevent backflow (reflux), especially during pressure fluctuations or patient movement. Reflux not only reduces treatment efficacy by limiting drug delivery but can also lead to unintended exposure of healthy brain tissue to the therapeutic agent, potentially causing side effects.

Also, optimal CED catheters need to have small radius but be able to target deep tissue structures. Conventional CED catheters developed to overcome this often necessitate patients to be under general anesthesia in an operating room during the infusion process, due to their rigidity and potential for causing discomfort, or possibly under an MRI (magnetic resonance imaging) scanner for the whole duration of the treatment. This adds complexity and cost to the procedure and may not be suitable for all patients. There is a need for improved catheter designs that address these and other issues.

### SUMMARY

In one or more embodiments, a medical catheter includes a multi-lumen tube comprising a proximal end, a distal end, a first lumen, and a second lumen that is independent of the first lumen. The first lumen is sealed at the distal end. The catheter further includes a retractable (removable) stylet carried in the first lumen of the multi-lumen tube. The stylet is adapted to stiffen the multi-lumen tube during a placement process and is movable relative to the multi-lumen tube. The first lumen being sealed at the distal end prevents the retractable stylet from protruding from the distal end of the multi-lumen tube. The first lumen being sealed forms a hermetic barrier that vents only proximally through a hub of the catheter and limits entrainable gas volume to microliter order under high pressure (e.g., entrainable gas volume is limited to <0.5 microliters under, e.g., 300 mm Hg). The catheter further includes a capillary tube of the second lumen of the multi-lumen tube. The capillary tube includes a distal tip for releasing an agent at the distal tip to a biological tissue. The distal tip of the capillary tube is an exposed portion of the capillary tube that protrudes beyond the distal end of the multi-lumen tube.

In one or more embodiments, a catheter includes a tube comprising a proximal end, a distal end, and a lumen. The lumen is sealed at the distal end. The catheter further includes a retractable stylet carried in the lumen, wherein the retractable stylet is adapted to stiffen the tube during a placement process and is movable relative to the tube, wherein the lumen being sealed at the distal end prevents the retractable stylet from protruding from the distal end of the tube. The catheter further includes a coupler connected to the distal end of the tube. And the catheter further includes a capillary tube carried in the lumen, the capillary tube comprising a distal tip for releasing an agent at the distal tip to a biological tissue, wherein the distal tip of the capillary tube is an exposed portion of the capillary tube that protrudes beyond the distal end of the tube and a distal end of the coupler.

In one or more embodiments, a convection-enhanced delivery (CED) method includes a plurality of steps. The steps include a step of placing a distal tip of a catheter at a target location in the brain. The catheter includes a multi-lumen tube comprising a proximal end, a distal end, a first lumen, and a second lumen that is independent of the first lumen, the first lumen being sealed at the distal end. The catheter further includes a retractable stylet carried in the first lumen of the multi-lumen tube, wherein the stylet is adapted to stiffen the multi-lumen tube during a placement process and is movable relative to the multi-lumen tube, wherein the first lumen being sealed at the distal end prevents the retractable stylet from protruding from the distal end of the multi-lumen tube. The catheter further includes a capillary tube of the second lumen of the multi-lumen tube, the capillary tube comprising the distal tip for releasing an agent at the distal tip to a biological tissue, wherein the distal tip of the capillary tube is an exposed portion of the capillary tube that protrudes beyond the distal end of the multi-lumen tube. The steps of the method further include a step of removing the retractable stylet at least partially from the first lumen to impart flexibility to the catheter, and a step of delivering an agent through the capillary tube to the target location. In one or more embodiments, the catheter may further include a third lumen containing one or more sensors, such as sensors configured to detect reflux, distribution of the therapeutic agent, infusion pressure, temperature, catheter location, electrical impedance, or chemical concentration. Data from the sensor(s) may be transmitted to a control unit that automatically adjusts infusion parameters to optimize drug delivery and minimize reflux. In another embodiment, a method of convection-enhanced delivery includes placing the catheter at a target location, retracting the stylet to impart flexibility, delivering the agent through the capillary tube, and maintaining a seal between the catheter and brain tissue to reduce reflux during infusion. The method may further include detecting infusion parameters via the sensor(s), adjusting infusion parameters based on the detected data, and performing infusion under stereotactic guidance and conscious sedation.

### BRIEF DESCRIPTION OF DRAWINGS

The disclosed embodiments have other advantages and features which will be more readily apparent from the detailed description, the appended claims, and the accompanying figures (or drawings). A brief introduction of the figures is below.

FIG. 1A is a schematic diagram showing a process of placing a conventional catheter in brain tissue.

FIG. 1B is a schematic diagram showing reflux during a process of infusing a drug in brain tissue with a conventional catheter.

FIG. 1C is a schematic diagram showing reflux during a process of infusing a drug in brain tissue with a conventional catheter.

FIG. 1D is a schematic diagram showing convection during a process of infusing a drug in brain tissue with a conventional catheter.

FIG. 2 is a schematic diagram showing a side view of a catheter for CED therapy, in accordance with one or more embodiments.

FIG. 3 is a schematic diagram showing a side view of a hub of a catheter for CED therapy, in accordance with one or more embodiments.

FIG. 4 is a schematic diagram showing a perspective view of a stylet after it is removed from a catheter for CED therapy, in accordance with one or more embodiments.

FIG. 5 is a schematic diagram showing a sectional view of a multi-lumen tube of a catheter for CED therapy, in accordance with one or more embodiments.

FIG. 6 is a schematic diagram showing a side view of a distal portion of a catheter for CED therapy, in accordance with one or more embodiments.

FIG. 7 is a schematic diagram showing a perspective view of a distal portion of a catheter for CED therapy, in accordance with one or more embodiments.

FIG. 8 is a schematic diagram showing a side view of a reinforcing member at a distal portion of a catheter for CED therapy, in accordance with one or more embodiments.

FIG. 9 is a schematic diagram showing a sectional view of a multi-lumen tube of a catheter for CED therapy, in accordance with one or more embodiments.

FIG. 10 is a flow chart illustrating a process for delivering a therapeutic agent to brain tissue, in accordance with one or more embodiments.

### DETAILED DESCRIPTION

The Figures (FIGS.) and the following description relate to preferred embodiments by way of illustration only. It should be noted that from the following discussion, alternative embodiments of the structures and methods disclosed herein will be readily recognized as viable alternatives that may be employed without departing from the principles of what is claimed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

In the following description, for purposes of explanation, numerous specific details are set forth to provide a thorough understanding of the inventive concept. In the interest of clarity, not all features of an actual implementation are described. Moreover, the language used in this disclosure has been principally selected for readability and instructional purposes, and may not have been selected to delineate or circumscribe the inventive subject matter, resort to the claims being necessary to determine such inventive subject matter. Reference in this disclosure to “one embodiment” or to “an embodiment” or “another embodiment” means that a particular feature, structure, or characteristic described in connection with the embodiment is included in at least one embodiment of the invention, and multiple references to “one embodiment” or “an embodiment” or “another embodiment” should not be understood as necessarily all referring to the same embodiment.

The terms “a,” “an,” and “the” are not intended to refer to a singular entity unless explicitly so defined but include the general class of which a specific example may be used for illustration. The use of the terms “a” or “an” may therefore mean any number that is at least one, including “one,” “one or more,” “at least one,” and “one or more than one.” The term “or” means any of the alternatives and any combination of the alternatives, including all the alternatives, unless the alternatives are explicitly indicated as mutually exclusive. The phrase “at least one of” when combined with a list of items, means a single item from the list or any combination of items in the list. The phrase does not require all the listed items unless explicitly so defined.

### Configuration Overview

This disclosure pertains to a convection-enhanced delivery (CED) catheter designed to address limitations associated with conventional catheter designs. Techniques disclosed herein look to overcome challenges related to air bubbles, reflux, and the need for general anesthesia and MRI scanners during CED procedures. In one or more embodiments, the disclosed catheter is an invasive catheter that features a multi-lumen design, incorporating two primary lumens: a stylet lumen and an agent delivery lumen. In one or more embodiments, the disclosed catheter is an invasive catheter that features a multi-lumen design, incorporating three or more primary lumens: a stylet lumen, an agent delivery lumen, and one or more lumens to carry additional elements such as sensors, waveguides, or electrodes to the distal end.

The stylet lumen houses a retractable stylet, providing rigidity during insertion into a subject's tissue, such as the brain parenchyma. In one or more embodiments, the stylet lumen houses the stylet and is sealed at a distal portion of the multi-lumen tube. As a result, when the stylet is removed from the lumen, the distal seal prevents any air bubbles from migrating into the biological tissue. In one or more embodiments, the stylet lumen is dedicated to the stylet and is not in fluid communication with other lumens.

In one or more embodiments, the agent-delivery lumen is used for the delivery of an agent. The agent may be a therapeutic agent (e.g., drug), a pharmaceutical composition, a nutritional composition, and the like. In one or more embodiments, the agent-delivery lumen is dedicated for agent delivery and is not in fluid communication with other lumens. In one or more embodiments, the agent-delivery lumen may include a capillary tube that extends continuously from the proximal end or Luer lock adapter to the distal tip of the catheter. The distal portion of the catheter may include a coupler connected to a distal end of the multi-lumen tube created of a different or same material for contact with tissue and delivery of agent.

The catheter may gradually reduce in diameter towards the distal tip of the catheter so that the area at which the agent enters the biological tissue is minimized. The gradual change in diameter may take the form of stepped shape, tapering shape, and/or any suitable combination. By way of examples, the catheter may take the shape of multiple steps that gradually reduce the diameters of the catheter towards the distal tip and minimize reflux. A first step at the distal tip of the capillary tube may be defined between the capillary tube and a reinforcement sleeve that may be coated with hydrophilic material (e.g., hydrogel) to improve a seal with brain tissue and minimize reflux. The reinforcement sleeve may be made of metal (e.g., magnetic resonance imaging-compatible metal) to impart rigidity to the distal tip of catheter during insertion.

A second step further upstream from the first step may be defined between the reinforcement sleeve and a coupler (distal adapter) that connects the reinforcement sleeve with the multi-lumen tube. The stylet lumen may be designed such that when housing the stylet, the stylet may be inserted furthermost to a position that is proximal to and upstream of the coupler. As a result, since the stylet lumen is separate from the agent-delivery lumen and since the stylet lumen is sealed off upstream of the coupler, air that may be present in the stylet lumen cannot migrate past the coupler and into the brain tissue. The step design of the catheter and the coating on the reinforcement sleeve help minimize the risk of reflux and improve targeted drug delivery.

Upon reaching the target location within the tissue, the stylet can be withdrawn from the stylet lumen. This renders the catheter flexible, conforming to the tissue's natural contours (e.g., brain) and minimizing the potential for tissue damage during manipulation or drug infusion. The ability to remove the stylet eliminates the need for general anesthesia, potentially allowing for CED procedures under conscious sedation or local anesthesia, improving the subject's comfort and accessibility. The catheter may be secured to tissue at the skull (bone screw) or skin to prevent migration and easy removal after treatment.

Overall, the present disclosure provides a CED catheter that addresses critical limitations of conventional designs. The multi-lumen configuration with a sealed stylet lumen prevents air bubble formation, the step design starting from the distal tip of the catheter and surface coating (or other mechanism like an expansile outer member) minimizes reflux, and the removable stylet enhances flexibility during infusion, potentially reducing the need for general anesthesia, allowing patient to be mobile during infusion and allowing infusion outside of the operating room, while patient is awake. These advancements contribute to a safer, more efficient, and potentially less invasive CED procedure.

Example Reflux and Convection with a Conventional Catheter

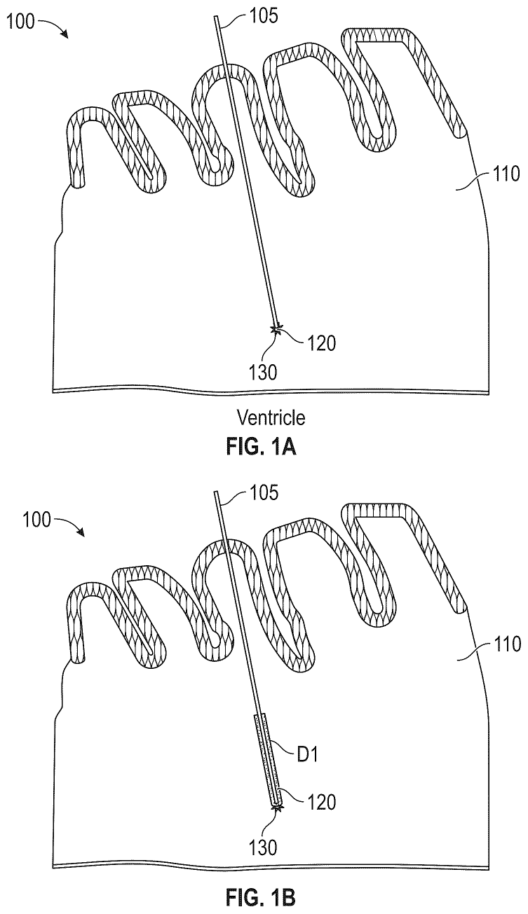

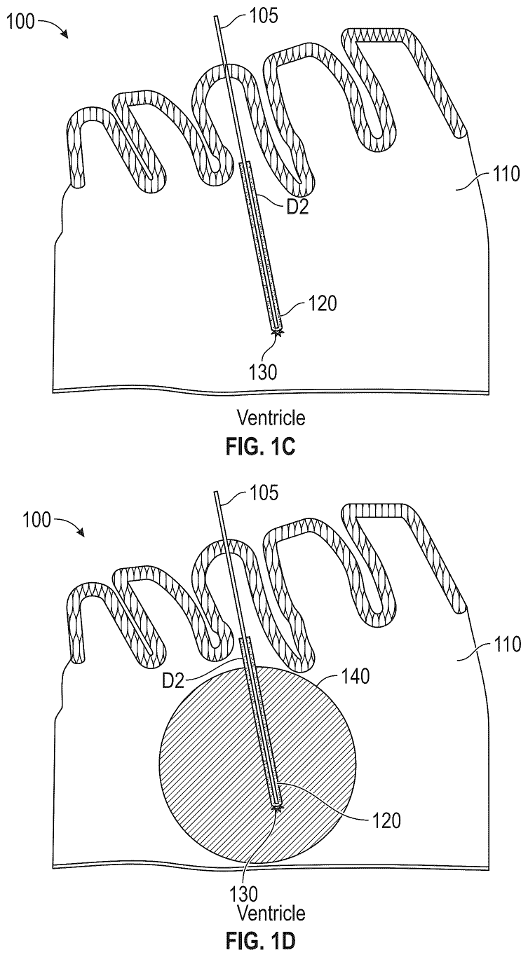

FIGS. 1A-1D illustrate the concepts of reflux and convection in CED therapy. FIG. 1A is a schematic diagram 100 showing a process of placing a conventional catheter 105 in brain tissue 110. Current stereotactic devices may be used to drill a hole and insert a bone screw into the skull. The bone screw defines a portal into the brain. The catheter 105 may be passed through the bone screw with seal and maneuvered to place a distal tip 120 of the catheter 105 at a target location 130 (e.g., 3-dimensional location having predetermined coordinates) in the brain tissue 110. Once the catheter 105 is positioned at the target location 130, the bone screw is tightened, which then secures the catheter 105 in place. After the placing process shown in FIG. 1A is complete, a process of infusing an agent (e.g., therapeutic agent) as shown in FIGS. 1B-1D may be started. While the brain tissue 110 is used as an example of a biological tissue, various embodiments of catheters described in this disclosure may be used for agent delivery to other suitable biological tissues.

FIGS. 1B-1C are schematic diagrams 100 showing reflux during a process of infusing an agent in the brain tissue 110 with the catheter 105. FIG. 1B shows that as the fluid carrying the therapeutic agent is infused from the catheter tip 120, the fluid leaks back out and up along the external surface of the catheter 105, instead of going outward into the brain tissue 110 (D1 in FIG. 1B). This is called reflux. Reflux occurs because the force required to push the fluid into the brain tissue, which is solid, may initially be greater than the force required to cause the fluid to flow up along the cavity created by the catheter 105 tube. Pressure is force per unit area, and as the area covered with the therapeutic fluid is built up and the reflux distance increases from D1 in FIG. 1B to D2 in FIG. 1C, the pressure around the catheter seal and the brain equilibrates. After reaching the equilibrium state shown in FIG. 1C, the therapeutic agent starts to convect as shown at 140 in FIG. 1D, go radially outward into the brain tissue 110. It is desirable to reduce the reflux distance D2 to improve treatment efficacy, avoid drug delivery to healthy brain tissue, and minimize loss of drug volume.

### Example Catheter for CED Therapy

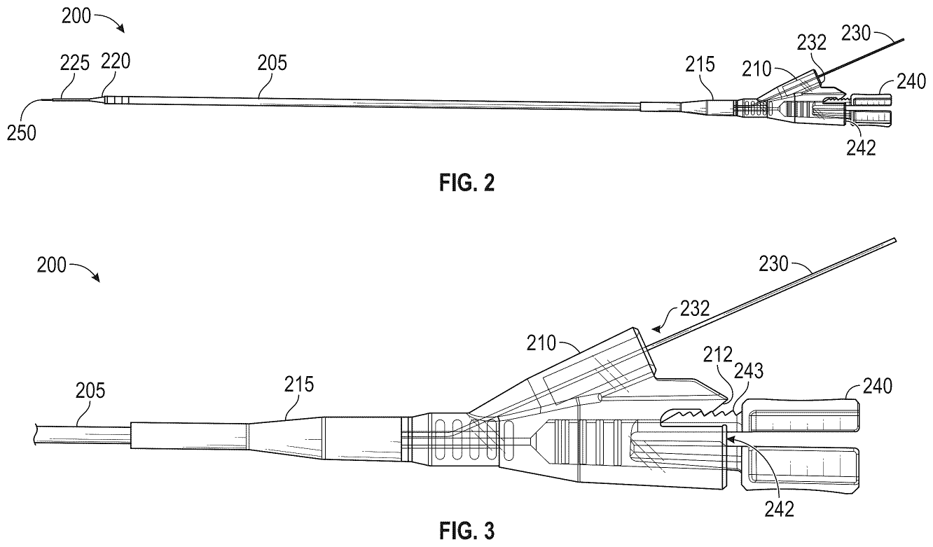

FIG. 2 is a schematic diagram showing a side view of a catheter 200 for CED therapy, in accordance with one or more embodiments. FIG. 3 is a schematic diagram showing a side view of a catheter hub 210 of the catheter 200, in accordance with one or more embodiments. FIGS. 2 and 3 show that the catheter 200 includes a multi-lumen tube 205, a catheter hub 210, a connecting member 215 (e.g., strain relief), a coupler 220, a reinforcement sleeve 225, a capillary tube 230, and a stylet 240. In other embodiments, the catheter 200 may exclude one or more of the components shown here. For example, the catheter 200 may exclude the reinforcement sleeve 225.

The multi-lumen tube 205 has a proximal end that is coupled to the catheter hub 210 via the connecting member 215. A distal end of the multi-lumen tube 205 is connected to the coupler 220. The multi-lumen tube 205 may be constructed from a biocompatible and flexible material. For example, the multi-lumen tube 205 may be constructed from polyamide (Pebax, Nylon), polyimide (PI), polyethylene (HDPE, LDPE), polyurethane or silicone, or from a polymer such as polyether ether ketone (PEEK) or a combination of these materials. A diameter of the multi-lumen tube 205 is narrow to minimize invasiveness while allowing sufficient internal space for the two or more lumens (e.g., agent-delivery lumen, stylet lumen) defined within. Although the illustrated embodiments show the multi-lumen tube 205 as having two lumens this is not intended to be limiting. In one or more embodiments, the multi-lumen tube 205 may define three or more lumens within a tube wall (e.g., sensor lumen in addition to agent-delivery lumen and stylet lumen).

In one or more embodiments, the total length of the multi-lumen tube 205 may be about in the range of about 1 and 16 inches. In one or more embodiments, the largest diameter of the multi-lumen tube 205 may be about in the range of about 500 and 5000 microns. For example, the total length of the multi-lumen tube 205 may be about 6 inches, and the largest diameter of the multi-lumen tube 205 may be about 1800 microns.

Although embodiments of the catheter 200 are described as including the multi-lumen tube 205 having two or more lumens, this may not always be the case. In one or more embodiments, the catheter 200 may include a tube which defines the main body of the catheter 200, where the tube defines a single-lumen through which the capillary tube 230 and the stylet 240 extend. For example, instead of having separate lumens for the capillary tube 230 and the stylet 240, the catheter 200 in accordance with one or more embodiments may include a single lumen through which at least the capillary tube 230, and the stylet 240 extend, where the stylet 240 is removable from the single lumen, and where the distal end of the single lumen tube terminates such that no air from the single lumen can escape out of the distal end of the catheter 200.

The connecting member 215 (e.g., strain relief) may be a sleeve or an adapter that attaches the multi-lumen tube 205 to the catheter hub 210. In one or more embodiments, the multi-lumen tube 205 is bonded to the catheter hub 210, and the connecting member 215 is a heat shrink that covers portions of both the lumen tube 205 and the catheter hub. Alternately, the connecting member 215 is a molded component that covers a portion of the multi-lumen tube 205 and that snaps into the catheter hub 210. The catheter hub 210 serves as the external connection point for the catheter 200. The catheter hub 210 may include shafts that are in fluid communication with respective lumens of the multi-lumen tube 205. In the embodiment shown in FIGS. 2 and 3, the catheter hub 210 includes a first shaft 232 that is in fluid communication with an agent-delivery lumen of the multi-lumen tube 205, and a second shaft 242 that is in fluid communication with a stylet lumen of the multi-lumen tube 205. The catheter hub 210 may be made of a rigid, or semi-rigid, transparent material or may otherwise have a clear visualization window integrated into a hub wall, allowing for visual confirmation of, e.g., fluid flow within the agent-delivery lumen without presence of any air bubbles.

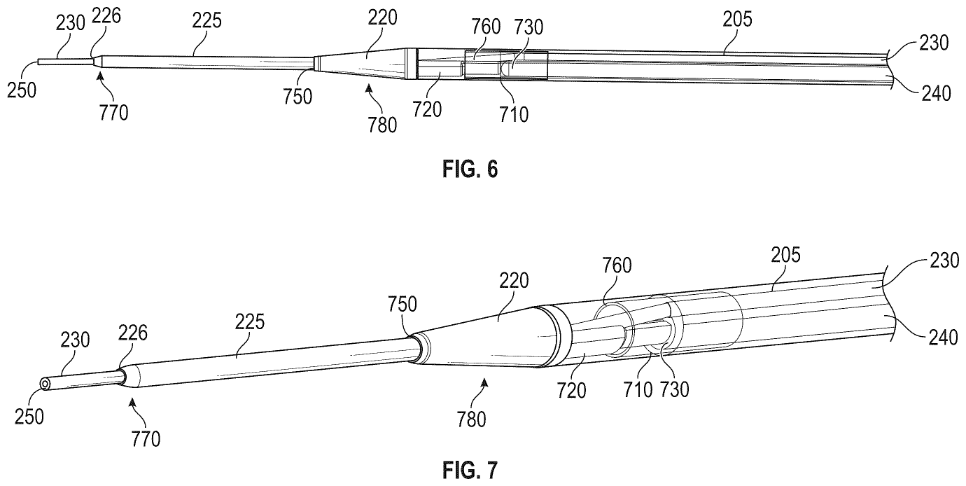

The coupler 220 may be connected to the distal end of the multi-lumen tube 205 and may limit a portion along a length of the catheter 200 through which the stylet 240 may extend. The reinforcement sleeve 225 may be connected to a distal end of the coupler 220. The coupler 220 has a conical shape and defines a step between the reinforcement sleeve 225 having a first diameter and the multi-lumen tube having a second diameter that is greater than the first diameter. For example, an outer diameter of the reinforcement sleeve may be in the range of about 0.1 to 0.2 millimeters, and an outer diameter of the multi-lumen tube may be in the range of about 1 to 3 millimeters. In one or more embodiments, an outer diameter of the reinforcement sleeve may be about 1 millimeters and an outer diameter of the multi-lumen tube may be about 3 millimeters. Additional configuration details of the distal portion of the catheter 200 including the coupler 220 and the reinforcement sleeve 225 will be provided in connection with FIGS. 6-8. The step between the reinforcement sleeve 225 and the multi-lumen tube 205 may be a tapered portion, as shown in the drawings. Alternately, or in addition, the step may be an abrupt step (e.g., 90 degree angle) between the reinforcement sleeve 225 and the multi-lumen tube 205.

FIGS. 2 and 3 further show that the capillary tube 230 passes through the first shaft 232 of the catheter hub 210 and through a first lumen (agent-delivery lumen) of the multi-lumen tube 205, and the stylet 240 passes through the second shaft 242 of the catheter hub 210 and through a second lumen (stylet lumen) of the multi-lumen tube 205.

In one or more embodiments, the capillary tube 230 is a therapeutic agent delivery tube that is continuous from a Luer lock adapter (not shown) to a distal tip 250 of the catheter 200. The Luer lock adapter may be connected to an infusion pump (not shown) that pumps fluid including a target agent through the capillary tube 230 under pressure. A total length of the continuous capillary tube 230 from the distal tip 250 to the infusion pump may be in the order of several inches or several feet. For example, the total length may be in the range of about 2 inches to 10 feet.

The capillary tube 230 of the catheter 200 may be made of a material that is biocompatible and flexible. The material may be chosen such that it won't cause any adverse reactions or toxicity within the brain tissue. For example, the capillary tube 230 may be made of polyurethane that offers a good balance of flexibility and strength, making it suitable for navigating the brain without kinking or breaking. As another example, the capillary tube 230 may be made of silicone that is highly flexible and biocompatible, thereby minimizing tissue damage during insertion. As another example, the capillary tube 230 may be made of PEEK or a biocompatible plastic. In one or more embodiments, the capillary tube 230 may be made of fused silica, polyimide, silastic or any combination of suitable materials.

The flexibility may enable the capillary tube 230 to conform to brain contours, allowing for easy insertion of the flexible tip to a precise 3-dimensional location in the brain without causing excessive tissue damage. An outer diameter of the capillary tube 230 may be in the order of micrometers. Generally, the smaller the diameter of the capillary tube, the better the seal of the catheter with the surrounding tissue, resulting in better drug distribution and less reflux. As a result, it is advantageous to have a smaller diameter for the capillary tube 230. For example, the outer diameter of the capillary tube 230 may be in the range of about 100 micrometers to 2000 micrometers. In one or more embodiments, the outer diameter of the capillary tube 230 at the distal tip may be about 500 micrometers.

The capillary tube 230 may be primed prior to or during insertion so that there is no void space or air bubbles in the agent-delivery lumen that may be pushed into the brain tissue during infusion. Since the diameter of the capillary tube 230 is small (e.g., about 500 micrometers) a total volume of the capillary tube 230 may be relatively small (e.g., about 6 microliters). As a result, dead volume is minimized and a very small amount of the drug is lost due to the priming process, which may be advantageous in certain types of treatments like gene therapy, where the cost of the drug may be very high. Priming the system prevents air bubbles from being introduced, and since the outer diameter of the capillary tube 230 is relatively small, the drug lost during the process is minimal. Another advantage of having the outer diameter of the capillary tube 230 in the stated range is a better seal between the outer surface of the catheter and the brain tissue, which reduces reflux (e.g., a shorter reflux distance) and improves convection. This in turn allows for increased pressure at the infusion pump, thereby increasing the infusion rate per minute and reducing the total infusion treatment time.

To provide rigidity for navigating the (flexible) distal tip 250 of the catheter 200 during insertion to a target location in the brain, the catheter 200 includes the stylet 240 that is accommodated in a dedicated stylet lumen of the multi-lumen tube 205 during insertion. The stylet 240 extends through a stylet lumen of the multi-lumen tube 205 and is adapted to stiffen the catheter 200 during the placing process. The stylet 240 is retractable (e.g., partially or entirely removable) for drug delivery, allowing the flexible multi-lumen tube 205 and the drug delivery tube 230 to conform to brain contours.

The stylet 240 may be made of a rigid material that imparts stiffness to the catheter 200 during insertion when the stylet 240 is accommodated in the stylet lumen of the multi-lumen tube 205. The stylet 240 material should be stiff enough to offer good control and stability while navigating the catheter 200 through brain tissue while preventing deflection, buckling or bending during insertion, ensuring an optimal insertion path for minimal tissue disruption. For example, the stylet 240 may be made of rigid or semi-rigid plastic, MRI-compatible metal or alloy, stainless steel, tungsten, titanium, PEEK, ceramic, glass and the like.

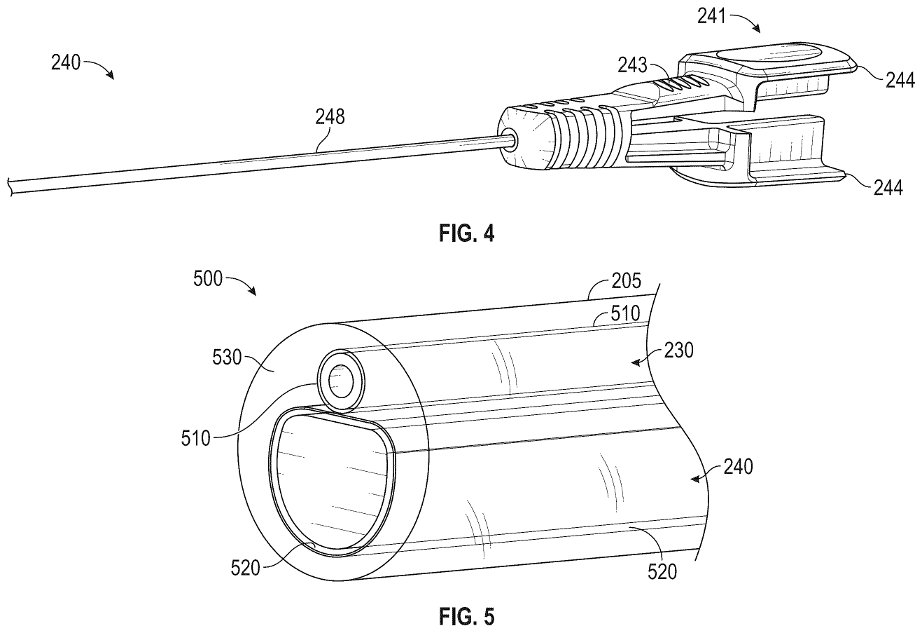

FIG. 4 shows the stylet 240 after it is removed from the catheter hub. As shown in FIGS. 2-4, the stylet 240 may have a handle 241 at a distal end and a main body 248 that is integrally formed with the handle 241. The handle 241 may include arms 244 having grooves 243 on an external surface that are adapted to engage with one or more ridges 212 formed in the stylet shaft 242 of the catheter hub 210. The arms 244 may be biased to remain engaged with the ridge 212 such that the stylet 240 remains locked in place in the stylet lumen of the multi-lumen tube 205 and in the stylet shaft 242 of the catheter hub 210 before and during the placing operation. An operator may compress the two arms 244 toward each other to release the engagement of the grooves 243 with the ridge 212, allowing the operator to either reposition the stylet 240 in the stylet lumen of the multi-lumen tube 205 or to remove the stylet 240 from the catheter 200 completely, as shown in FIG. 4. Other embodiments may employ a stylet having a different configuration for precisely placing the catheter 200 in target tissue. An outer diameter of the stylet 240 may be in the order of 1 mm. For example, the outer diameter of the stylet 240 may be in the range of about 0.5 millimeters to 3 millimeters. In one or more embodiments, the outer diameter of the stylet 240 may be about 1.5 millimeters. A total length of the stylet may be in the range of about 25 millimeters to 500 millimeters. In one or more embodiments, an outer diameter of the stylet 240 may be about 0.1 inches.

FIG. 5 shows a sectional view 500 of the multi-lumen tube 205, in accordance with one or more embodiments. As shown in FIG. 5, the multi-lumen tube 205 defines two distinct lumens (channels or shafts) 510, 520 running longitudinally within tube wall 530. Although FIG. 5 shows the multi-lumen tube 205 as having two lumens 510, 520, this is not intended to be limiting. In one or more embodiments, the multi-lumen tube 205 may define three or more lumens within the tube wall 530.

In the embodiment shown in FIG. 5, the first lumen 510 is an agent-delivery lumen that houses the capillary tube 230 for delivering a therapeutic agent to a patient during drug infusion and the second lumen 520 is a stylet lumen for accommodating the stylet 240 to impart rigidity to the catheter 200 during the placing process. FIG. 5 shows that to minimize dead space, the inner diameter of the first lumen 510 is about the same as the outer diameter of the capillary tube 230, and the inner diameter of the second lumen 520 is about the same as the outer diameter of the stylet 240.

Although embodiments of the catheter 200 are described as including the agent-delivery lumen 510 and the capillary tube 230 are separate components wherein the capillary tube 230 is a continuous tube that passes through the lumen 510 of the multi-lumen tube 205, this may not always be the case. In one or more embodiments, the capillary tube 230 and the agent-delivery lumen 510 may be integrally formed into a single component. For example, the lumen 510 may itself define at least a portion of the capillary tube 230 such that there is no separate tube passing through the lumen 510. A tube may be coupled or integrally formed at the proximal and/or distal ends of the multi-lumen tube 205 to define one or both portions of the capillary tube 230 that extend from the proximal and/or distal ends of the multi-lumen tube 205.

FIGS. 6-8 are schematic views describing the configuration of a distal portion of the catheter 200, in accordance with one or more embodiments. In the embodiments shown in FIGS. 6-8, the distal portion of the catheter 200 includes at least the coupler 220, the reinforcement sleeve 225, and the distal tip 250 of the capillary tube 230.

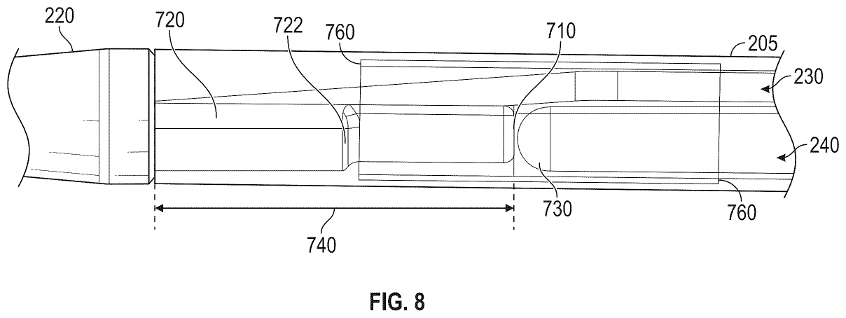

FIGS. 6-8 show that the multi-lumen tube 205 is attached to the coupler 220 at its distal end. The coupler 220 may be made of a rigid or semi-rigid material (e.g., plastic, PEEK, MRI-compatible metal, and the like). The distal end of the multi-lumen tube 205 and/or the coupler 220 act as a seal that prevent any air in the stylet lumen (520 in FIG. 5) escaping from the catheter 200 into brain tissue. That is, as shown most clearly in FIGS. 7 and 8, the stylet lumen may be sealed off within the tube wall (530 in FIG. 5) at a position 710 that is proximal to and upstream of the coupler 220. The stylet lumen 520 being sealed forms a hermetic barrier that vents only proximally through the catheter hub 210 and limits entrainable gas volume in the microliter order under high pressure (e.g., entrainable gas volume is limited to <0.5 microliters under, e.g., 300 mm Hg).

Although embodiments of the catheter 200 are described as including the coupler 220 that is a separate component attached to the multi-lumen tube 205, this may not always be the case. In one or more embodiments, the catheter 200 may include a tapered distal portion instead of the coupler 220, where the tapered distal portion is integrally formed with the multi-lumen tube 205.

FIGS. 6-8 also show that the coupler 220 includes a projecting member 720 that extends upstream of the coupler 220 and terminates at the sealed end of the stylet lumen at the position 710. The projecting member 720 may be adapted to reinforce the sealed end of the stylet lumen and prevent the distal end 730 of the stylet 240 from advancing any further in the stylet lumen of the multi-lumen tube 205 when the operator is pushing on the stylet 240 and the catheter 200 during the placing process. The stylet lumen may allow exit of air in the lumen from the catheter hub 210 at the proximal end when the stylet 240 is pushed into the lumen. That is, the stylet 240 may extend into the stylet lumen toward the coupler 220 until the distal end 730 of the stylet 240 contacts the projecting member 720 at the position 710 and cannot extend any further into the multi-lumen tube 205. Any air in the stylet lumen displaced by the advancing distal end 730 of the stylet 240 is pushed back toward the proximal end of the catheter 200 and out of the stylet shaft 242 of the catheter hub 210. Any air in the stylet lumen is thus prevented from being forced out into the tissue since the stylet lumen is sealed off within the tube walls and does not have any air outlet other than the opening of the stylet shaft 242 of the catheter hub 210 at the proximal end.

A portion 740 of the multi-lumen tube 205 downstream of the dead end of the stylet lumen at the position 710 may transition from a tube having multiple lumens (510, 520 in FIG. 5) into a tube having a single lumen within the tube wall, the single lumen corresponding to an agent-delivery lumen through which the capillary tube 230 may extend. As shown in FIGS. 6-8, the agent-delivery lumen in the portion 740 may extend at an angle to make the lumen and the capillary tube 230 within the lumen coaxial with the coupler 220 at a distal end 750 of the coupler 220.

FIGS. 6-8 also show that the tube wall at the distal portion of the multi-lumen tube 205 may include a reinforcing member 760. The reinforcing member 760 may be a cylindrical member that is adapted to stiffen the portion 740 and a region that overlaps with the distal end 730 of the stylet 240 when the distal end 730 is positioned in contact with the projecting member 720. For example, the reinforcing member 760 may be made of a metal such as titanium or a thermoplastic. Any material or composite that is suitable for medical use and has the appropriate properties (e.g., stiffness) may be used for manufacturing the reinforcing member 760. The reinforcing member 760 may be designed to prevent buckling and kinking at the position 710 where the distal tip 730 of the stylet 240 is positioned.

As shown most clearly in FIG. 8, the reinforcing member 760 may overlap at least partially with the projecting member 720 and also overlap at least partially with the distal end 730 of the stylet 240. The projecting member 720 may have a groove 722 to axially accommodate the cylindrical reinforcing member 760 while outer dimensions of the multi-lumen tube 205 remain unchanged. Since the stylet 240 terminates at the position 710 in the multi-lumen tube 205, which is more flexible than the stylet 240, the reinforcing member 760 imparts the necessary rigidity to ensure the distal portion of the catheter 200 has a desired stiffness gradient that is suitable for inserting and maneuvering the catheter 200 to a desired location within the brain with precision.

In one or more embodiments, as shown in FIGS. 6-7, the distal portion of the catheter 200 also includes the reinforcement sleeve 225. The reinforcement sleeve 225 is connected to the distal end 750 of the coupler 220. In some embodiments, the reinforcement sleeve 225 and the coupler 220 may be formed integrally. In other embodiments, the reinforcement sleeve 225 may be attached to the distal end 750 of the coupler 220 to form an airtight seal. In yet other embodiments, the reinforcement sleeve 225 may be omitted and the properties of the underlying capillary tube 230 may be modified to have the desired stiffness gradient imparted by the combination of the reinforcement sleeve 225 with the distal tip 250 axially extending from it. The reinforcement sleeve 225 may be made of any material that imparts stiffness to minimize deflection during the placing process. For example, the reinforcement sleeve 225 may be a metallic tube. As another example, the reinforcement sleeve 225 may be made of a thermoplastic or PEEK. Other similar materials or composites may also be used.

The reinforcement sleeve 225 is sealed and mated with the distal end of the agent-delivery lumen (510 in FIG. 5) of the multi-lumen tube 205 and extends the agent-delivery lumen distal to the coupler 220. The capillary tube 230 may extend though the agent-delivery lumen in the multi-lumen tube 205, in the coupler 220 and in the reinforcement sleeve 225 as a continuous tube. The reinforcement sleeve 225 surrounds a portion of the capillary tube 230 that protrudes beyond the distal end 750 of the coupler 220. That is, a portion including the distal tip 250 of the capillary tube 230 may protrude from a distal end 226 of the reinforcement sleeve 225 by a predetermined distance. The predetermined distance may be the range of about 1 millimeter to 50 millimeters. In one or more embodiments, the predetermined distance by which the capillary tube 230 extends from the distal end 226 of the reinforcement sleeve 225 to the distal tip 250 may be about 10 millimeters. The reinforcement sleeve 225 at the distal end 226 may be fused to the outer surface capillary tube 260 to create an airtight seal.

A length of the reinforcement sleeve 225 between the distal end 226 of the reinforcement sleeve 225 and the distal end 750 of the coupler 220 may be in the range of about 2 millimeters to 300 millimeters. In one or more embodiments, the length may be about 20 millimeters.

The reinforcement sleeve 225 may include a tapered portion 770 at the distal end 226. The tapered portion 770 defines a first step of dilation between the capillary tube 230 having an outer diameter that is less than an outer diameter of the reinforcement sleeve 225. During infusion, the drug flows out of the distal tip 250 of the catheter 200 and due to reflux, flows around and back against the outer surface of the capillary tube 230 until it reaches the tapered portion or step 770. The dilation at the tapered portion 770 helps create a better seal to the surrounding tissue, preventing the drug from flowing further back up against the outer surface of the reinforcement sleeve 225, thereby limiting the reflux distance to a length of the capillary tube 230 between the distal tip 250 and the distal end 226 of the reinforcement sleeve 225. Also, the infused fluid carrying the therapeutic agent hitting the tapered step 770 may create a turbulent flow that helps to accentuate the drug distribution by pushing the fluid outwards into the brain tissue and convect into the desired region of the brain. The step 770 may be a tapered portion, as shown in the drawings. Alternately, or in addition, the step may be an abrupt step (e.g., 90 degree angle) between the capillary tube 230 and the reinforcement sleeve 225.

FIGS. 6-8 also show that the coupler 220 has a tapered or conical shape that defines a second step 780 of dilation between the reinforcement sleeve 225 having an outer diameter that is less than an outer diameter of the multi-lumen tube 205. Similar to the first step of dilation between the capillary tube 230 and the reinforcement sleeve 225, the second step of dilation 780 also helps create a better seal to the surrounding tissue, preventing the drug from flowing back up against the outer surface of the guide tube 205, creating an additional backstop to prevent reflux and accentuate drug distribution into the brain tissue with a desired shape and in the desired area. Although FIGS. 6-7 illustrate an embodiment in which the catheter 200 has one step along the reinforcement sleeve 225, other embodiments may include two or more steps of successive larger dilations along a length of the catheter 200.

At least a portion of the outer surface of the reinforcement sleeve 225 may be treated with a hydrophilic coating to improve the seal with the brain tissue and further prevent reflux. Hydrophilic coatings such as hydrogel attract and absorb water. When applied to the catheter's 200 outer surface, the hydrogel coating swells upon contact with bodily fluids, creating a tighter and more secure seal between the catheter 200 and the surrounding tissue. This seal minimizes reflux of the infused drug, ensuring that the medication reaches its intended target. For example, at least a portion of the outer surface of the reinforcement sleeve 225 may be treated with a hydrogel-swell band. In some embodiments, the hydrogel-swell band may be made of a material that causes a 10-20% increase in its outside diameter within 60-90 seconds. The band may create a reflux threshold pressure that may be provided as input to a closed-loop system associated with the catheter 200 that may generate and regulate infusion pressure.

Mechanisms other than or in addition to hydrophilic coatings may also be employed to improve the seal and prevent reflux. Such mechanisms may define an expansile outer member that is adapted to have a first diameter during the placing process and a second diameter during an infusion process, the second diameter being greater than the first diameter. The expansile outer member may be utilized alternately or in addition to the reinforcement sleeve 225 for preventing reflux. In some embodiments, a shape morphism actuation of the expansile outer member may be at 32-35 degrees Celsius with saline concentration of 0.9% or the shape morphism actuation of the expansile outer member may be by the infusate. For example, shape memory polymers designed to change shape in response to body temperature can be used, potentially improving the seal as they conform to the tissue. Biocompatible adhesives (organic compounds, inorganic compounds) can be also used to create a stronger bond between the catheter and tissue. Mechanical mechanisms such as balloon catheters or expandable tip catheters may be employed as well. For example, the reinforcement sleeve 225 may have one or more balloons at the distal end 226 that can be inflated to create a secure seal. As another example, the reinforcement sleeve 225 may have an expandable tip at the distal end 226 that can be deployed to improve the seal.

In one or more embodiments, the catheter 200 may include one or more sensors to provide a closed self-sufficient system for controlled drug infusion. The sensors may be configured to measure signals at one or more positions along a length the catheter 200. Based on the measured sensor data, a control unit that may be included in the catheter (e.g., in the hub 210) or in equipment connected to the catheter (e.g., at the infusion pump) may detect various conditions related to catheter placement or drug infusion such as drug reflux, drug distribution, or treatment effect.

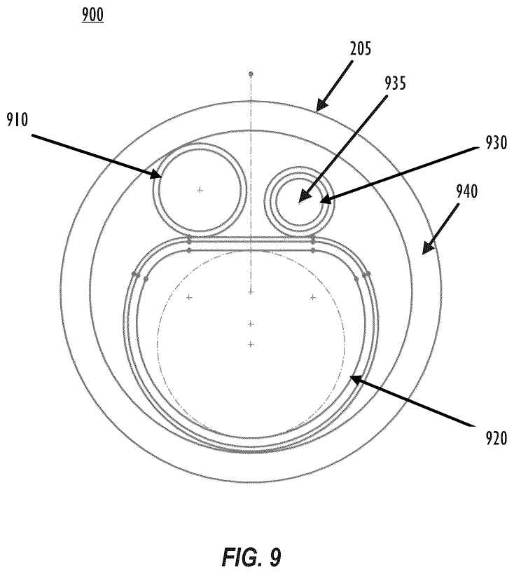

In one or more embodiments, the multi-lumen tube may include additional lumens (e.g., a third lumen (not shown) in addition to lumens 510, 520 within the tube wall 530 in FIG. 5) that may be dedicated to wiring or waveguides (e.g., fiber optic filament) for the sensor(s) and the catheter 200 may include components (e.g., electrodes) at various positions along the length of the catheter (e.g., at the reinforcement sleeve 225) to detect sensor data. For example, the sensor may be a pressure sensor configured to detect a pressure at the distal tip 250 or at the distal end 226 of the reinforcement sleeve 225, in-line, or at the infusion pump. The pressure sensor data may be used by a controller to adjust the infusion rate during the infusion process. For example, a controller may be able to detect at what pressure does the seal between the catheter 200 and the tissue finally break and drive the infusion rate as high as possible based on this information, thereby shortening the duration of the infusion treatment, or decreasing/halting infusion if a sudden change in pressure occurs. FIG. 9 illustrates a sectional view 900 of the multi-lumen tube 205, in accordance with one or more embodiments. As shown in FIG. 9, the multi-lumen tube 205 defines three distinct lumens (channels or shafts) 910 (e.g., for the capillary tube 230), 920 (e.g., for the stylet 240), and 930 running longitudinally within tube wall 940. Lumen 930 may include a pressure sensor 935 for monitoring pressure at, e.g., the distal tip of the catheter.

As another example, the sensor may be an electrical sensor to measure impedance or electrical parameters. In embodiments where the reinforcement sleeve 225 is a metallic tube, the electrical sensor may be implemented by carving out portions of the metallic tube and introduce one or more electrodes embedded within the reinforcement sleeve 225 such that they are flush with the external surface of the reinforcement sleeve. An impedance reading can then be obtained using the one or more electrodes in the reinforcement sleeve 225 and optionally, ground.

As yet another example, one or more electrodes may be placed on one or more surfaces at a distal end of the catheter. The electrodes may be placed as rings or as segments along the distal end and extending back along a portion or an entire length of the catheter. The electrodes may measure impedance, electrical activity, or chemical activity. The measurement may be utilized by a control system of the catheter to control one or more parameters during an infusion operation. Alternately, or in addition, the measurement data may be utilized after the infusion operation to determine characteristics of the infusion.

As yet another example, the sensor may be a positional sensor (e.g., coil) to measure deflection. As yet another example, the sensor may be a temperature sensor to measure a temperature at one or more positions along the catheter. Yet another example, the sensor may be a chemical or electrolyte sensor to detect a level of concentration of a chemical or electrolyte at one or more positions along the catheter. For example, the chemical sensor may be utilized to detect locations along the catheter where the drug is contacting the catheter to detect a reflux distance. Another example is a chemical sensor to monitor the therapeutic outcome of the infused agent. Yet another example, the sensor may be a positioning sensor (e.g., electromagnetic, sonographic) to determine the precise location of the distal tip of the catheter. For example, an electromagnetic position sensor (e.g., a position tracker manufactured by POLHEMUS INC.) may determine the precise location of the distal tip without the need for medical imaging like X-ray or MRI. Yet another example, an ultrasound transducer to image the tissue in the region outside the catheter.

In one or more embodiments, based on the sensor data, a controller may detect one or more properties during an infusion process, and control one or more parameters of the infusion process based on the detected one or more properties. For example, a controller at the infusion pump may dynamically control and maximize the infusion rate and when leakage or reflux is detected, manipulate the infusion rate to be able to slow down, get a better seal again, detect the improved seal, and then ramp the infusion rate back up again while continuously monitoring the sensor data.

In some embodiments, the catheter 200 may further comprise a third lumen (not shown in the drawings) that houses one or more sensors configured to provide real-time infusion data. The sensors may include one or more of: a reflux sensor, an agent distribution sensor, a pressure sensor, a temperature sensor, a positioning sensor, an electrical impedance sensor, or a chemical concentration sensor. Sensor outputs may be transmitted to a control unit that may be integrated into the infusion pump or into the catheter hub 210, to automatically adjust infusion parameters such as rate, pressure, or duration. In this way, the system may provide a closed-loop infusion control that reduces the likelihood of reflux and maximizes therapeutic efficiency.

In some embodiments, a method of using the catheter 200 may include placing the distal tip 250 under stereotactic guidance, retracting the stylet 240 from the stylet lumen 520 to impart flexibility, and delivering a therapeutic agent through the capillary tube 230 while maintaining a tissue seal (e.g., at the reinforcement sleeve 225). The infusion may be performed under conscious sedation, allowing patient comfort without requiring general anesthesia. Exemplary therapeutic agents include proteins, liposomal nanoparticles, viral or non-viral gene therapy vectors, chemotherapeutics, or combinations thereof. The stepwise diameter changes of the catheter 200, such as those defined between the capillary tube 230 and the reinforcement sleeve 225 (step 770), and between the reinforcement sleeve 225 and the multi-lumen tube 205 (step 780), together with respective hydrophilic coatings or expansile members, may limit reflux distance to less than 10 mm during infusion.

Prior to placement, the capillary tube 230 may be primed to minimize dead volume, for example to less than about 10 microliters, thereby conserving therapeutic material in high-value treatments such as gene therapy. During infusion, the control unit may use the measured sensor data from, e.g., a pressure sensor, electrodes embedded in the reinforcement sleeve 225, or other sensors positioned in third lumen, to enforce infusion rate limits, trigger alarms to alert the operator, or automatically modulate infusion to minimize drug loss while maximizing infusion efficiency.

### Example Method of Delivering a Therapeutic Agent to Brain Tissue

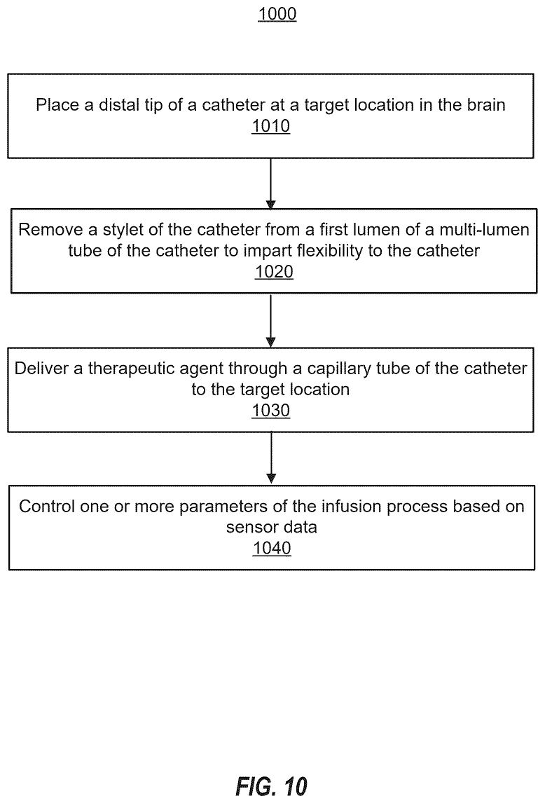

FIG. 10 is a flow chart illustrating a process 1000 for delivering a therapeutic agent to brain tissue, in accordance with one or more embodiments.

The method 1000 begins with an operator placing 1010 a distal tip of a catheter (e.g., catheter 200 of FIGS. 2-9) at a target location in the brain. For example, as shown in FIGS. 2, 6, 7, since the catheter has a stiffness gradient such that the distal tip 250 is flexible while the reinforcement sleeve 225, the coupler 220, and the multi-lumen tube 205 with the reinforcing member 760 and the stylet 240 in a stylet lumen are more rigid, the operator can maneuver and guide the distal tip to a target location in the brain without deflection.

After guiding the distal tip to the target location, the operator may remove 1020 the stylet from the catheter (e.g., from lumen 510 in FIG. 5) to impart flexibility to the catheter. Since the stylet is removed, the multi-lumen tube now becomes relatively more flexible. As a result, the patient can remain more mobile and need not be in the operating room during subsequent drug infusion.

The operator may then deliver 1030 a therapeutic agent through a capillary tube (e.g., 230 in FIGS. 2, 3, 5-8) of the catheter to the target location. Since the catheter may include the step design with one or more steps (e.g., step 770 in FIG. 7), since an external surface of the catheter may be subject to, e.g., a hydrophilic coating at one or more locations, and/or since the catheter may include an expansile outer member, the catheter may prevent reflux by creating a seal between brain tissue and the outer surface of the catheter. As a result, the drug delivery can be precisely targeted to achieve a desired distribution with minimal reflux. Also, infusion rate can be increased without breaking the seal between catheter and tissue.

The catheter may further include one or more sensors (e.g., pressure sensor 935 of FIG. 9) and a control system may (manually, semi-automatically, or fully autonomously) control 1040 one or more parameters (e.g., infusion rate) of the infusion process based on sensor data (e.g., data indicating one or more of a reflux condition, a distribution condition, a brain density metric, an infusion location, and the like) to optimize the infusion process.

### Additional Configuration Considerations

The foregoing description of the embodiments has been presented for the purpose of illustration; it is not intended to be exhaustive or to limit the patent rights to the precise forms disclosed. Persons skilled in the relevant art can appreciate that many modifications and variations are possible in light of the above disclosure.

At least one embodiment is disclosed and variations, combinations, and/or modifications of the embodiment(s) and/or features of the embodiment(s) made by a person having ordinary skill in the art are within the scope of the disclosure. Alternative embodiments that result from combining, integrating, and/or omitting features of the embodiment(s) are also within the scope of the disclosure. Where numerical ranges or limitations are expressly stated, such express ranges or limitations may be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). The use of the term “about” means±10% of the subsequent number, unless otherwise stated.

Use of the term “optionally” with respect to any element of a claim means that the element is required, or alternatively, the element is not required, both alternatives being within the scope of the claim. Use of broader terms such as comprises, includes, and having may be understood to provide support for narrower terms such as consisting of, consisting essentially of, and comprised substantially of. Accordingly, the scope of protection is not limited by the description set out above but is defined by the claims that follow, that scope including all equivalents of the subject matter of the claims. Each and every claim is incorporated as further disclosure into the specification and the claims are embodiment(s) of the present disclosure.

While several embodiments have been provided in the present disclosure, it should be understood that the disclosed systems and methods might be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted, or not implemented.

In addition, techniques, systems, subsystems, and methods described and illustrated in the various embodiments as discrete or separate may be combined or integrated with other systems, modules, techniques, or methods without departing from the scope of the present disclosure. Other items shown or discussed as coupled or directly coupled or communicating with each other may be indirectly coupled or communicating through some interface, device, or intermediate component whether electrically, mechanically, or otherwise.

Some portions of this description describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like.

Furthermore, it has also proven convenient at times, to refer to these arrangements of operations as modules, without loss of generality. The described operations and their associated modules may be embodied in software, firmware, hardware, or any combinations thereof.

Embodiments may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Finally, the language used in the specification has been principally selected for readability and instructional purposes, and it may not have been selected to delineate or circumscribe the patent rights. Many other embodiments will be apparent to those of skill in the art upon reviewing the above description. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights, which is set forth in the following claims.

What is claimed is:

1. A medical catheter, comprising:
a multi-lumen tube comprising a proximal end, a distal end, a first lumen, and a second lumen that is independent of the first lumen, the first lumen being sealed at the distal end;
a retractable stylet carried in the first lumen of the multi-lumen tube, wherein the stylet is adapted to stiffen the multi-lumen tube during a placement process and is movable relative to the multi-lumen tube, wherein the first lumen being sealed at the distal end prevents the retractable stylet from protruding from the distal end of the multi-lumen tube; and
a capillary tube of the second lumen of the multi-lumen tube, the capillary tube comprising a distal tip for releasing an agent at the distal tip to a biological tissue, wherein the distal tip of the capillary tube is an exposed portion of the capillary tube that protrudes beyond the distal end of the multi-lumen tube.

2. The medical catheter of claim 1, wherein the stylet is adapted to be retracted at least partially from the first lumen after the distal tip of the capillary tube is positioned at a desired position during the placement process to impart flexibility to the catheter during infusion.

3. The medical catheter of claim 1, wherein a diameter of the capillary tube is in a range of 100 micrometers to 2000 micrometers.

4. The medical catheter of claim 1, further comprising:
a coupler connected to the distal end of the multi-lumen tube; and
a reinforcement sleeve connected to the coupler, wherein the coupler has a conical shape and defines a step between the reinforcement sleeve having a first outer diameter and the multi-lumen tube having a second outer diameter that is greater than the first outer diameter.

5. The medical catheter of claim 4, wherein at least a portion of an external surface of the reinforcement sleeve is coated with a hydrophilic material.

6. The medical catheter of claim 4, wherein the reinforcement sleeve surrounds a portion of the capillary tube that protrudes beyond the distal end of the multi-lumen tube and the distal tip of the capillary tube extends through the reinforcement sleeve and protrudes from the reinforcement sleeve.

7. The medical catheter of claim 6, wherein reinforcement sleeve includes a tapered or stepped portion, the tapered or stepped portion defining a step between the reinforcement sleeve having the first outer diameter and the capillary tube having a third outer diameter that is less than the first outer diameter.

8. The medical catheter of claim 7, wherein a length of the capillary tube between the distal tip and a distal end of the reinforcement sleeve is in a range of 1 millimeter to 50 millimeters.

9. The medical catheter of claim 1, further comprising:
a reinforcing member disposed in the multi-lumen tube at a position where the first lumen terminates, wherein the first lumen is sealed at a position where the reinforcing member is located.

10. The medical catheter of claim 9, wherein the reinforcing member is a cylindrical member that is adapted to stiffen a portion of the multi-lumen tube where the first lumen is sealed.

11. The medical catheter of claim 1, further comprising:
a third lumen extending through the multi-lumen tube; and
at least one sensor in the third lumen for detecting one or more of an agent reflux, an agent distribution, or a treatment effect.

12. The medical catheter of claim 11, wherein the at least one sensor includes:
a pressure sensor configured to measure a pressure at one or more positions along the catheter;
an electrical sensor configured to measure an electrical impedance;
a temperature sensor configured to measure temperature at one or more positions along the catheter;
a chemical sensor configured to detect a level of concentration of a chemical at one or more positions along the catheter;
a positioning sensor to determine a location of the distal tip; or
an ultrasound transducer to image tissue in a region outside the catheter.

13. The medical catheter of claim 1, further comprising:
an expansile outer member downstream of the distal end of the multi-lumen tube, wherein the expansile outer member has a first outer diameter during a placing process and a second outer diameter during an agent delivery process, the second outer diameter being greater than the first outer diameter.

14. The medical catheter of claim 1, wherein the capillary tube extends continuously from a connector at the proximal end to the distal tip and passes through the second lumen of the multi-lumen tube.

15. A catheter, comprising:
a tube comprising a proximal end, a distal end, and a lumen, the lumen being sealed at the distal end;
a retractable stylet carried in the lumen, wherein the retractable stylet is adapted to stiffen the tube during a placement process and is movable relative to the tube, wherein the lumen being sealed at the distal end prevents the retractable stylet from protruding from the distal end of the tube;
a coupler connected to the distal end of the tube; and
a capillary tube carried in the lumen, the capillary tube comprising a distal tip for releasing an agent at the distal tip to a biological tissue, wherein the distal tip of the capillary tube is an exposed portion of the capillary tube that protrudes beyond the distal end of the tube and a distal end of the coupler.

16. A convection-enhanced delivery (CED) method, comprising:
placing a distal tip of a catheter at a target location in the brain, the catheter comprising:
a multi-lumen tube comprising a proximal end, a distal end, a first lumen, and a second lumen that is independent of the first lumen, the first lumen being sealed at the distal end;
a retractable stylet carried in the first lumen of the multi-lumen tube, wherein the stylet is adapted to stiffen the multi-lumen tube during a placement process and is movable relative to the multi-lumen tube, wherein the first lumen being sealed at the distal end prevents the retractable stylet from protruding from the distal end of the multi-lumen tube; and
a capillary tube of the second lumen of the multi-lumen tube, the capillary tube comprising the distal tip for releasing an agent at the distal tip to a biological tissue, wherein the distal tip of the capillary tube is an exposed portion of the capillary tube that protrudes beyond the distal end of the multi-lumen tube,
removing the retractable stylet at least partially from the first lumen to impart flexibility to the catheter; and
delivering an agent through the capillary tube to the target location.

17. The CED method of claim 16, further comprising:
preventing reflux by creating a seal between brain tissue and an outer surface in a distal portion of the catheter.

18. The CED method of claim 16, wherein the catheter includes a sensor in a third lumen of the multi-lumen tube for detecting one or more of a drug reflux, a drug distribution, location, or a treatment effect, and wherein the CED method further comprises:
detecting one or more properties during an agent delivery process using data obtained from the sensor; and
controlling one or more parameters of the agent delivery process based on the detected one or more properties.

19. The CED method of claim 16, wherein a diameter of the capillary tube is in a range of 100 micrometers to 2000 micrometers.

20. The CED method of claim 16, wherein the catheter further comprises:
a coupler connected to the distal end of the multi-lumen tube; and
a reinforcement sleeve connected to the coupler, wherein the coupler has a conical shape and defines a step between the reinforcement sleeve having a first outer diameter and the multi-lumen tube having a second outer diameter that is greater than the first outer diameter,
wherein an exterior surface of the reinforcement sleeve is coated with a hydrophilic material.
